(19) United States
(12) Patent Application Publication
(10) Pub. No.: US 2024/0316066 A1
(43) Pub. Date: Sep. 26, 2024

(54) COMPOSITION COMPRISING PIP4K2C INHIBITOR AS ACTIVE INGREDIENT FOR TREATMENT OF CANCER

(71) Applicant: GWANGJU INSTITUTE OF SCIENCE AND TECHNOLOGY, Gwangju (KR)

(72) Inventors: Darren WILLIAMS, Gwangju (KR); Hyunju LEE, Gwangju (KR); Da-Woon JUNG, Gwangju (KR); Eun-Mi JANG, Gwangju (KR); Jihee SOH, Gwangju (KR)

(21) Appl. No.: 18/676,998

(22) Filed: May 29, 2024

Related U.S. Application Data
(63) Continuation-in-part of application No. PCT/KR2022/019051, filed on Nov. 29, 2022.

(30) Foreign Application Priority Data
Nov. 30, 2021 (KR) 10-2021-0168747

(51) Int. Cl. A61K 31/58; A61K 31/192; A61K 31/47; A61K 31/506; A61K 31/5375; A61K 45/06; A61P 35/00; C12Q 1/6886; G01N 33/50

(52) U.S. Cl. CPC A61K 31/58; A61K 31/192; A61K 31/47; A61K 31/506; A61K 31/5375; A61K 45/06; A61P 35/00; C12Q 1/6886; G01N 33/5011; G01N 33/5023; G01N 2333/91215

(57) ABSTRACT

The present invention relates to a pharmaceutical composition for preventing or treating cancer comprising a PIP4K2C inhibitor as an active ingredient. The present invention provides a composition for efficient and radical treatment of cancer by discovering PIP4K2C as a new treatment target for cancer, which exhibits a high recurrence rate and is difficult to radically treat, and selecting a drug that inhibits PIP4K2C among known anticancer drugs.

### CROSS-REFERENCE TO RELATED APPLICATIONS

This application is a continuation in part of International Patent Application no. PCT/KR2022/019051, filed Nov. 29, 2022, which claims the benefit of priority of Korean Patent Application no. 10-2021-0168747, filed Nov. 30, 2021, each of which is hereby incorporated herein by reference in its entirety.

### TECHNICAL FIELD

The present invention relates to the discovery of PIP4K2C as a novel cancer treatment target and a composition of treating cancer using the same.

### BACKGROUND ART

Cancer is one of the diseases that accounts for the largest cause of death in modern people. Cancer is a disease caused by changes in normal cells due to gene mutations that result from various causes, and refers to malignant tumors that do not follow the differentiation, proliferation, and growth patterns of normal cells. Cancer is characterized by “uncontrolled cell growth”. This abnormal cell growth causes formation of a cell mass called a tumor, which infiltrates surrounding tissues and, in severe cases, may metastasize to other organs of the body. Cancer is an intractable chronic disease that is not radically cured in many cases even if it is treated with surgery, radiotherapy, chemotherapy, and the like, causes pain to patients, and ultimately leads to death. In particular, in recent years, the global cancer incidence rate has increased by 5% or higher every year due to increased elderly population, environmental deterioration, or the like. According to the WHO report, it is estimated that within the next 25 years, the number of cancer patients will increase to 30 million, of which 20 million will die from cancer.

Rhabdomyosarcoma is an undifferentiated tumor that occurs in the muscles of the human body and can appear throughout the body. It occurs mainly in children and adolescents and accounts for about 60 to 70% of all soft tissue sarcomas. Symptoms thereof vary depending on the location of the tumor, and the exact cause thereof is unknown. Histologically, rhabdomyosarcoma is predominantly divided into embryonic and alveolar types. Among these types, the alveolar type accounts for 20 to 25% of rhabdomyosarcoma and has a poor prognosis because it has a gene fusion of PAX3/7-FOXO1.

For treatment of rhabdomyosarcoma, various methods such as surgery, radiotherapy, and chemotherapy are performed in combination, and patient-specific therapy is performed depending on the location of the tumor or the presence of gene translocation. However, the 5-year survival rate for relapsed or metastatic rhabdomyosarcoma is very low at 20 to 30%, and thus there is a need to develop a new and more effective therapeutic agent. Accordingly, the present invention has been made in order to solve the above problems.

### DISCLOSURE

### Technical Problem

The present inventors have made extensive research efforts to develop a new effective therapeutic agent for cancer that is difficult to radically cure and has a high recurrence rate. As a result, the present inventors have discovered for the first time the PIP4K2C gene as a new target for cancer treatment, and have found that, when the expression of PIP4K2C is inhibited, breast cancer, lung cancer, pancreatic cancer, colorectal cancer, and rhabdomyosarcoma cell lines are efficiently killed, suggesting that various PIP4K2C inhibitors are useful as compositions for the radical treatment of cancer.

Therefore, an object of the present invention is to provide a pharmaceutical composition, which is effective in treating cancer, by proposing a new target for treating cancer.

Other objects and advantages of the present invention will become more apparent from the following detailed description, the appended claims and the accompanying drawings.

However, objects to be achieved by the present invention are not limited to the above-mentioned object, and other objects not mentioned above may be clearly understood by those skilled in the art from the following description.

### Technical Solution

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present invention pertains.

Throughout the present specification, it is to be understood that when any part is referred to as “comprising” any component, it does not exclude other components, but may further comprise other components, unless otherwise specified.

According to one aspect of the present invention, the present invention provides a pharmaceutical composition for preventing or treating cancer comprising a PIP4K2C inhibitor as an active ingredient.

The present inventors have made extensive research efforts to develop a new effective therapeutic agent for cancer that is difficult to radically cure and has a high recurrence rate. As a result, the present inventors have discovered for the first time the PIP4K2C gene as a new target for cancer treatment, and have found that, when the expression of PIP4K2C is inhibited, breast cancer, lung cancer, pancreatic cancer, colorectal cancer, and rhabdomyosarcoma cell lines are efficiently killed, suggesting that various PIP4K2C inhibitors are useful as compositions for the radical treatment of cancer.

In the present invention, “cancer” refers to or describes the physiological condition typically characterized by unregulated cell growth in a mammal. In the present invention, the cancer to be prevented, ameliorated or treated may be a solid tumor consisting of a mass formed by abnormal cell growth in a solid organ. Depending on the location of the solid organ, the cancer may be gastric cancer, liver cancer, glioblastoma, ovarian cancer, colorectal cancer, head and neck cancer, bladder cancer, renal cell cancer, breast cancer, metastatic cancer, prostate cancer, rhabdomyosarcoma, pancreatic cancer, melanoma, lung cancer, or the like, without being limited thereto.

In the present invention, the term “rhabdomyosarcoma” refers to a tumor arising from striated muscle cells, which is a very aggressive form of cancer that arises from mesenchymal cells that have not differentiated into myocytes of skeletal muscle. Rhabdomyosarcoma is divided into four subtypes: embryonal rhabdomyosarcoma, alveolar rhabdomyosarcoma, pleomorphic rhabdomyosarcoma, and spindle-cell/sclerosing rhabdomyosarcoma. Rhabdomyosarcoma is generally considered a disease of childhood because it most often develops in people under the age of 18.

According to the present invention, “embryonal rhabdomyosarcoma” is a rare histological form of cancer of connective tissue wherein the mesenchymal-derived malignant cells resemble the primitive developing skeletal muscle of the embryo, and refers to the most common soft tissue sarcoma that occurs in children. Embryonal rhabdomyosarcoma is also known as fusion-negative rhabdomyosarcoma (FN-RMS).

According to the present invention, “alveolar rhabdomyosarcoma” refers to a subtype of the rhabdomyosarcoma soft tissue cancer family whose lineage is from mesenchymal cells and which is related to skeletal muscle cells. An alveolar rhabdomyosarcoma tumor is similar to the alveolar tissue of the lung, and although the location of the tumor varies from patient to patient, the tumor is generally found in the head and neck area, the urinary tract in men and women, torso, and extremities. Alveolar rhabdomyosarcoma in children and adolescents accounts for approximately 1% of all malignant tumors, has an incidence of one per million, and occurs sporadically without genetic predisposition in most cases.

According to the present invention, “pleomorphic rhabdomyosarcoma” refers to a rare soft tissue sarcoma characterized by occurring almost exclusively in adults, which is composed of bizarre polygonal, round, and spindle cells which display evidence of skeletal muscle differentiation. Patients usually present with a rapidly growing, painful mass located in the deep soft tissues of the extremities, but also other anatomic regions. Prognosis is generally poor.

According to the present invention, “botryoid rhabdomyosarcoma” is characterized in that it manifests as a polypoid or “grape-like lesion” beneath the mucosa, does not infiltrate the epithelium, and has an intratubular appearance. It is almost always found in mucosal-lined organs, including the vagina, bladder, and nasopharynx, and often presents in infants younger than a year old, as a round, grape-like mass on the affected organ. Histologically, cells of the botryoid variant are defined by a dense tumor layer under an epithelium.

In the present specification, the term “prevention” means inhibiting the occurrence of a disorder or disease in a subject who has never been diagnosed as having the disorder or disease, but is likely to suffer from such disorder or disease.

In the present specification, the term “treatment” means (a) inhibiting the progress of a disorder, disease or symptom; (b) alleviating the disorder, disease or symptom; or (c) eliminating the disorder, disease or symptom. When the composition of the present invention is administered to a subject, it functions to reduce the expression level or activity of PIP4K2C, thereby inhibiting the progress of a tumor, or eliminating the tumor, or alleviating the tumor. Thus, the composition of the present invention may serve as a therapeutic composition for this disease alone, or may be administered in combination with other anticancer pharmacological ingredients and applied as a therapeutic aid for cancer. Accordingly, as herein used, the term “treatment” or “therapeutic agent” is meant to encompass “treatment aid” or “therapeutic aid agent”.

In the present specification, the term “administration” or “administering” means administering a therapeutically effective amount of the composition of the present invention directly to a subject so that the same amount is formed in the subject's body.

In the present specification, the term “therapeutically effective amount” refers to an amount of the composition containing a pharmacological ingredient sufficient to provide a therapeutic or prophylactic effect to a subject to whom the pharmaceutical composition of the present invention is to be administered. Accordingly, the term “therapeutically effective amount” is meant to encompass a “prophylactically effective amount”.

In the composition according to a specific embodiment of the present invention, the cancer may be selected from the group consisting of ovarian cancer, rhabdomyosarcoma, colorectal cancer, pancreatic cancer, gastric cancer, liver cancer, breast cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, hematological cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, cancer of ureter, renal cell carcinoma, renal pelvic carcinoma, central nervous system (CNS) tumor, primary CNS lymphoma, spinal cord tumor, brainstem glioma, and pituitary adenoma.

In the composition according to a specific embodiment of the present invention, the PIP4K2C inhibitor may comprise an antibody or antigen-binding fragment thereof that specifically binds to PIP4K2C, an aptamer that specifically binds to PIP4K2C, a nucleic acid molecule that inhibits expression of a nucleotide encoding PIP4K2C, or a small molecule compound that inhibits expression of PIP4K2C.

According to a specific embodiment of the present invention, the PIP4K2C inhibitor is at least one small molecule compound selected from the group consisting of dutasteride, aprepitant, ivacaftor, adapalene, and pharmaceutically acceptable salts thereof.

According to another aspect of the present invention, the present invention provides a method for screening a composition for preventing or treating rhabdomyosarcoma comprising steps of:
(a) bringing a test substance into contact with a biological sample containing cells expressing PIP4K2C; and
(b) measuring the activity or expression level of the PIP4K2C or measuring the viability or proliferation rate of the cells expressing PIP4K2C,
wherein, when the activity or expression level of the PIP4K2C decreased or when the viability or proliferation rate of the cells expressing PIP4K2C decreased, the test substance is determined to be a composition for preventing or treating rhabdomyosarcoma.

In the present invention, the term “biological sample” refers to any sample containing cells expressing PIP4K2C, obtained from mammals, including humans, and includes, but is not limited to, tissues, organs, cells, or cell cultures.

In the method according to a specific embodiment of the present invention, the biological sample may be selected from the group consisting of whole blood, leukocytes, peripheral blood mononuclear cells, leukocyte buffy coat, plasma, serum, sputum, tears, mucus, nasal washes, nasal aspirate, breath, urine, semen, saliva, peritoneal washings, pelvic fluids, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, tissue, tissue-derived cells, cell extract, and cerebrospinal fluid, without being limited thereto.

The term “test substance” used while referring to the screening method of the present invention refers to an unknown substance that is added to a sample containing cells expressing PIP4K2C and used in screening to test whether it affects the activity or expression level of PIP4K2C. Examples of the test substance include, but are not limited to, compounds, nucleotides, peptides, and natural extracts. The step of measuring the expression level or activity of PIP4K2C in the biological sample treated with the test substance may be performed by various methods of measuring expression level and activity known in the art. As a result of the measurement, when the expression level or activity of PIP4K2C decreased, the test substance may be determined to be a composition for preventing or treating cancer.

In the present specification, the term “decrease in activity or expression level” refers to a decrease in the expression level or unique in vivo function or expression level of PIP4K2C measured in a test substance-administered group compared to that of PIP4K2C measured in a control group. The decrease in activity includes not only a simple decrease in function but also an ultimate decrease in activity due to a decrease in stability. Specifically, the term “decrease in activity or expression level” may mean a state in which the activity or expression level decreased by at least 20%, more specifically at least 40%, even more specifically at least 60%, compared to that in the control group.

According to still another aspect of the present invention, the present invention provides a composition for diagnosing cancer comprising, as an active ingredient, an agent for measuring the expression level of PIP4K2C protein or a gene encoding the same. According to the present invention, it has been discovered for the first time that there is a correlation between the PIP4K2C protein and rhabdomyosarcoma. Accordingly, when the PIP4K2C protein or the gene encoding the same is highly expressed in a subject, the subject is determined to have rhabdomyosarcoma.

In the present specification, the term “highly expressed” refers to a case where the expression level of the PIP4K2C protein or PIP4K2C gene is significantly higher than that in a healthy subject, and specifically, a case where the expression level is 30% or higher, more specifically 50% or higher, most specifically 70% or higher than that in a healthy subject.

In the present specification, the term “subject” refers to a subject that provides a sample for which the expression level of the PIP4K2C protein or the gene encoding the same is to be measured, and that is ultimately to be analyzed as to whether cancer has developed. Subjects include, without limitation, humans, mice, rats, guinea pigs, dogs, cats, horses, cows, pigs, monkeys, chimpanzees, baboons, or rhesus monkeys, and are specifically humans. Since the composition of the present invention provides information for not only diagnosing cancer at present but also predicting the genetic risk of being diagnosed with cancer in the future, the subject in the present invention may be a cancer patient or a healthy subject who has not yet developed cancer.

In the composition according to a specific embodiment of the present invention, the agent for measuring the expression level of PIP4K2C protein may be an antibody or antigen-binding fragment thereof that specifically binds to the PIP4K2C protein, an aptamer that specifically binds to PIP4K2C, or a small molecule compound that inhibits expression of PIP4K2C.

According to the present invention, the PIP4K2C protein in the present invention may be detected according to an immunoassay method using an antigen-antibody reaction and used to analyze whether the subject has developed cancer. This immunoassay may be performed according to various immunoassay or immunostaining protocols developed previously.

For example, when the method of the present invention is performed according to a radioimmunoassay method, antibodies labeled with radioisotopes (e.g., ¹⁴C, ¹²⁵I, ³²P and ³⁵S) may be used. In the present invention, the antibody that specifically recognizes the PIP4K2C protein is a polyclonal or monoclonal antibody, specifically, a monoclonal antibody.

The antibody in the present invention may be produced by methods commonly practiced in the art, for example, a fusion method, a recombinant DNA method, or a phage antibody library method.

Cancer may be predicted by analyzing the intensity of the final signal by the above-described immunoassay process. That is, when the signal for the PIP4K2C protein in the sample from the subject is stronger than that in a sample from a healthy subject, the subject is determined to have cancer.

According to the present invention, “antigen-binding fragment” refers to a portion of a polypeptide among the entire structure of an immunoglobulin, to which an antigen may bind. Examples of the antigen-binding fragment include, but are not limited to, F(ab′)2, Fab′, Fab, Fv, and scFv.

According to the present invention, “specifically binding” has the same meaning as “specifically recognizing”, and means that an antigen and an antibody (or a fragment thereof) specifically interact with each other through an immunological reaction.

In the present invention, an aptamer that specifically binds to the PIP4K2C protein may also be used instead of the antibody. In the present specification, the term “aptamer” refers to a single-stranded nucleic acid (RNA or DNA) molecule or peptide molecule that binds to a specific target substance with high affinity and specificity.

According to a specific embodiment of the present invention, the agent for measuring the expression level of the gene encoding the PIP4K2C protein is a primer or probe that specifically binds to the nucleic acid molecule of the gene.

In the present specification, the term “nucleic acid molecule” is meant to encompass DNA (gDNA and cDNA) and RNA molecules, and nucleotides, which are the basic structural units in nucleic acid molecules, include not only natural nucleotides, but also analogues having modified sugar or base moieties.

In the present specification, the term “primer” refers to an oligonucleotide which serves as a starting point for synthesis under conditions in which synthesis of a primer extension product complementary to a nucleic acid chain (template) is induced, i.e., in the presence of nucleotides and a polymerization agent such as DNA polymerase and at a suitable temperature and pH. Specifically, the primer is a deoxyribonucleotide single chain. The primers that are used in the present invention may include naturally occurring dNMPs (i.e., dAMP, dGMP, dCMP and dTMP), modified nucleotides or non-natural nucleotides. In addition, the primers may also include ribonucleotides.

The primer in the present invention may be an extension primer which is annealed to a target nucleic acid and forms a sequence complementary to the target nucleic acid by a template-dependent nucleic acid polymerase. The extension primer is extended to the location where an immobilized probe is annealed and occupies the area at which the probe is annealed.

The extension primer that is used in the present invention includes a hybridizing nucleotide sequence complementary to a specific nucleotide sequence of a target nucleic acid, e.g., the PIP4K2C gene. The term “complementary” means that a primer or a probe is complementary enough to selectively hybridize to a target nucleic acid sequence under a certain annealing or hybridization condition. The term “complementary” is meant to encompass both “substantially complementary” and “perfectly complementary”. Specifically, it means perfectly complementary. In the present specification, the term “substantially complementary sequence” is meant to include not only a completely matching sequence but also a sequence partially mismatching with a specific sequence to be compared within a range where it can anneal to the sequence and serve as a primer.

The primer should be long enough to prime the synthesis of an extension product in the presence of a polymerase. Although the suitable length of the primer is determined depending on a number of factors, such as temperature, pH and the source of the primer, it is typically 15 to 30 nucleotides. Short primer molecules generally require lower temperatures to form a sufficiently stable hybrid complex with a template. The design of such a primer can be easily carried out by those skilled in the art with reference to a target nucleotide sequence using, for example, a program for primer design (e.g., PRIMER 3 program).

In the present specification, the term “probe” refers to a natural or modified monomer or a linear oligomer having linkages, which includes a deoxyribonucleotide and ribonucleotide that can be hybridized with a specific nucleotide sequence. Specifically, the probe is single-stranded for maximum efficiency in hybridization. More specifically, the probe is a deoxyribonucleotide. As the probe that is used in the present invention, a sequence perfectly complementary to a specific nucleotide sequence of the PIP4K2C gene may be used, but a sequence substantially complementary thereto may also be used as long as specific hybridization is not interrupted. In general, the stability of a duplex formed by hybridization tends to be determined by the match of sequences at terminals, and thus it is preferred to use a probe which is complementary to the 3′-end or 5′-end of the target sequence.

As used herein, the term “composition for diagnosing” refers to an integrated mixture or device comprising a means for measuring the expression level of the PIP4K2C protein or PIP4K2C gene in order to determine whether or not the subject has developed cancer or to predict the likelihood of developing cancer. Thus, this term may also be referred to as a “diagnostic kit”.

### Advantageous Effects

The features and advantages of the present invention are summarized as follows:
(a) The present invention provides a pharmaceutical composition for preventing or treating cancer comprising an inhibitor of PIP4K2C as an active ingredient.
(b) The present invention provides a composition for efficient and radical treatment of cancer not only by discovering PIP4K2C as a new treatment target for intractable cancer that is difficult to radically cure and has a high recurrence rate, but also by selecting drugs that inhibit PIP4K2C among known anticancer drugs.

### BRIEF DESCRIPTION OF DRAWINGS

FIGS. 1A and 1B show the results of MTT assay after treatment of SJCRH30 with FDA-approved drugs for 24 hours, according to one example of the present invention (*=p<0.05, **=p<0.01, ***=p<0.001).

FIGS. 2A and 2B show the results of MTT assay after treatment of SJCRH30 and CCD-18Co with ivacaftor for 24 hours, according to one experimental example of the present invention (*=p<0.05, **=p<0.01, ***=p<0.001).

FIG. 3 shows the results of MTT assay after treatment of SJCRH30 with pazopanib HCl and ivacaftor for 24 hours, according to one experimental example of the present invention (*=p<0.05, **=p<0.01, ***=p<0.001).

FIG. 4 shows the results of evaluating the anticancer effects of ivacaftor against colorectal cancer, pancreatic cancer, lung cancer, and breast cancer, according to one example of the present invention.

FIG. 5 shows the results of evaluating the anticancer effects of adapalene against colorectal cancer, pancreatic cancer, lung cancer, and breast cancer, according to one example of the present invention.

FIG. 6 shows the results of evaluating the anticancer effects of dutasteride against colorectal cancer, pancreatic cancer, and lung cancer, according to one example of the present invention.

### BEST MODE

The present invention aims at developing a new effective therapeutic agent for intractable cancer, which is difficult to radically treat previously and has a high recurrence rate. The present invention is for effective treatment of cancer using the expression level of a specific gene in a biological sample to be tested. According to the present invention, the PIP4K2C gene as a new target for cancer treatment has been discovered for the first time, and it has been found that when the expression of PIP4K2C is inhibited, breast cancer, lung cancer, pancreatic cancer, colorectal cancer, and rhabdomyosarcoma cell lines are efficiently killed, indicating that various PIP4K2C inhibitors may be provided to rhabdomyosarcoma patients as optimal therapeutic compositions for radical treatment of cancer.

### MODE FOR INVENTION

Hereinafter, the present invention will be described in more detail by way of examples. These examples are only for illustrating the present invention in more detail, and it will be obvious to those skilled in the art that the scope of the present invention according to the subject matter of the present invention is not limited by these examples.

### Examples

Hereinafter, a specific example of the algorithm of the present invention will be described in detail. Using the InfoGenomeR algorithm, genetic mutations were found in whole-genome sequencing data of rhabdomyosarcoma cell line (SJCRH30) samples, and genes with high expression levels were found in transcriptome (RNA sequencing) data. Thereafter, in order to discover candidate genes in rhabdomyosarcoma cell line samples, PIP4K2C, one of the genes having high gene expression level and integer copy number while having genetic mutations, was selected as a new target for rhabdomyosarcoma treatment. Finally, drug candidates highly reactive with the PIP4K2C gene were discovered from an FDA-approved drug library through the DeepAffinity algorithm.

### [Examples 1] Materials and Experimental Methods

### Example 1-1. Reagents

RPMI1640, penicillin, and streptomycin used for cell culture were purchased from GIBCO, and fetal bovine serum (FBS) was purchased from Invitrogen. MTT (3-(4,5-dimethylthiazol-2-yl)-2,5-diphenyltetrazolium bromide) for cell proliferation evaluation was purchased from Gold Biotechnology, and DMSO was purchased from Sigma-Aldrich. The FDA-approved drug library was purchased from Selleckchem.

### Example 1-2. Cell Culture

SJCRH30 human alveolar rhabdomyosarcoma cells were purchased from ATCC. Cells were cultured in RPMI1640 supplemented with 10% fetal bovine serum (FBS), 1% penicillin, and streptomycin. CCD-18Co human colon fibroblasts were purchased from the Korea Cell Line Bank. Cells were cultured in DMEM supplemented with 10% fetal bovine serum (FBS), 1% penicillin, and streptomycin. All cells were cultured in an incubator at 37° C. under 5% CO₂.

### Example 1-3. MTT (3-(4,5-Dimethylthiazol-2-yl)-2,5-Diphenyltetrazolium Bromide) Assay

Cell proliferation was evaluated through MTT analysis. SJCRH30 and CCD-18Co cells were seeded in triplicate in 96-well plates at a density of 2×10⁴ cells per well. After 24 hours, each FDA-approved drug was diluted in fetal bovine serum (FBS)-free medium and the cells were treated therewith. After 24 hours of incubation, the medium was replaced with MTT solution (0.5 mg/mL) and the plates were incubated at 37° C. under 5% CO₂. After 60 minutes of incubation, the supernatants were removed and 50 μL of DMSO was added to each well. The optical density in each well was measured at 570 nm using a microplate reader (VersaMax, Molecular Devices, USA).

### Example 1-4. Data Analysis

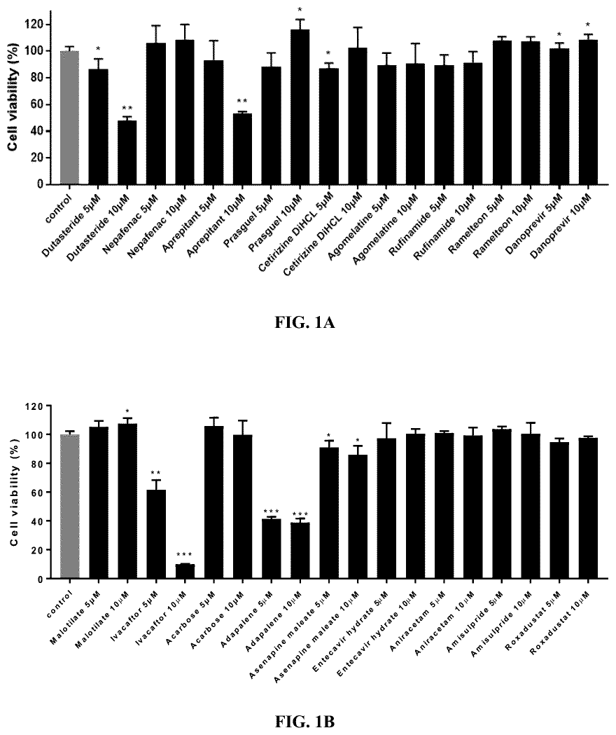

Student's t-test was used to determine statistical significance in FIGS. 1A-1B. A p value of less than 0.05 was considered statistically significant. Error bars are standard deviation.

### [Example 2] Evaluation of Anticancer Effects of FDA-Approved Drugs Against Rhabdomyosarcoma

MTT assay was performed to evaluate the effects of FDA-approved drugs on the proliferation of rhabdomyosarcoma cells (SJCRH30). Rhabdomyosarcoma cells were treated with each of 18 drugs at concentrations of 5 μM and 10 μM, respectively, and observed after 24 hours. A significant decrease in cell proliferation and a significant cell killing effect (10 μM) were found in the cells treated with each of dutasteride, aprepitant, ivacaftor, and adapalene (FIGS. 1A-1B).

### [Example 3] Evaluation of Selective Action of Ivacaftor on Rhabdomyosarcoma

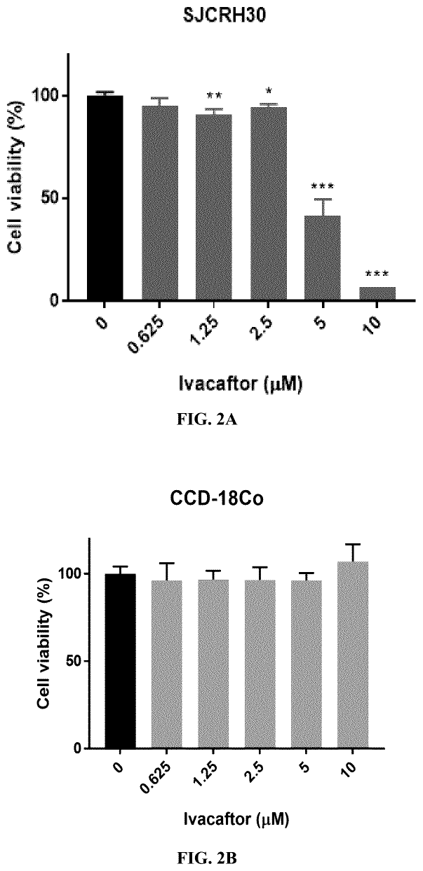

MTT assay was performed to evaluate the toxicity of ivacaftor, which exhibited the highest effect on cell proliferation reduction, to normal cells. Rhabdomyosarcoma cells (SJCRH30) and colon fibroblasts (CCD-18Co) were treated with ivacaftor at concentrations of 0.625 μM, 1.25 μM, 2.5 μM, 5 μM, and 10 UM and compared. When rhabdomyosarcoma cells (SJCRH30) were treated with ivacaftor, a significant decrease in the cell number was shown at 1.25 μM, 2.5 μM, 5 μM, and 10 μM, and when colon fibroblasts (CCD-18Co) were treated with ivacaftor, there was no significant decrease in the cell number at all of the concentrations. This indicates that ivacaftor does not exhibit toxicity in normal cells (FIGS. 2A-2B).

### [Example 4] Comparison of IC₅₀ Values of Pazopanib HCl and Ivacaftor

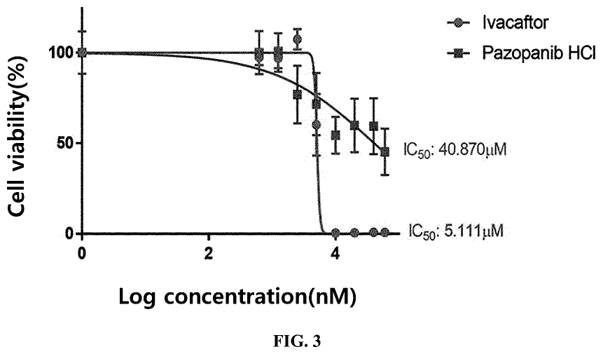

MTT assay was performed to compare IC₅₀ (half maximal inhibitory concentration) values of pazopanib HCl, which is used for soft tissue sarcoma patients, and ivacaftor, against rhabdomyosarcoma cells (SJCRH30). Rhabdomyosarcoma cells were treated with each of pazopanib HCl and ivacaftor at concentrations of 0.625 μM, 1.25 μM, 2.5 μM, 5 μM, 10 UM, 20 μM, 40 μM, and 60 UM for 24 hours and then observed. The IC₅₀ values of pazopanib HCl and ivacaftor were 40.870 μM and 5.111 μM, respectively, indicating that ivacaftor was more effective against rhabdomyosarcoma cells (SJCRH30) (FIG. 3).

### [Examples 5] Evaluation of Anticancer Effect Against Other Cancer Types

Among the drugs found to significantly reduce the cell proliferation of rhabdomyosarcoma cells (SJCRH30), the anticancer effects of ivacaftor, adapalene, and dutasteride against other cancer types were evaluated. MTT assay was performed by treating colorectal cancer cells (HCT-116), pancreatic cancer cells (PANC-1), lung cancer cells (A549), and breast cancer cells (MDA-MB-231) with each of ivacaftor and adapalene and treating colorectal cancer cells (HCT-116), pancreatic cancer cells (PANC-1), and lung cancer cells (A549) with dutasteride. The cells were treated with each of the three drugs at concentrations of 0.625 μM, 1.25 μM, 2.5 μM, 5 μM, and 10 UM for 24 hours and then observed.

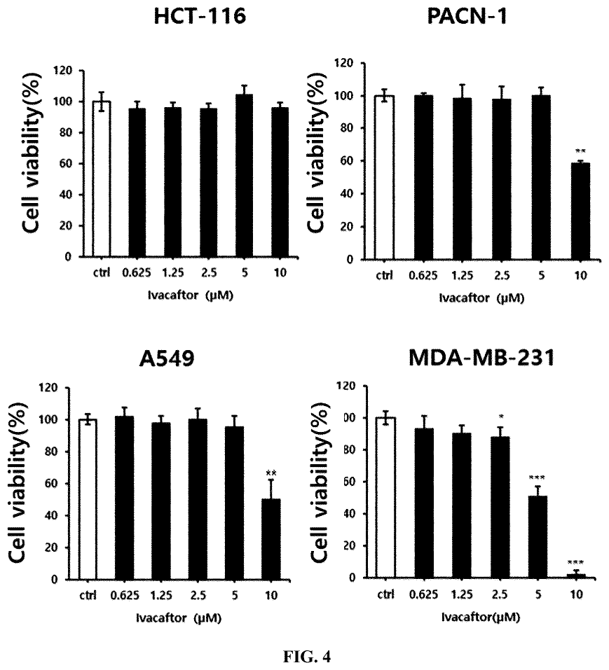

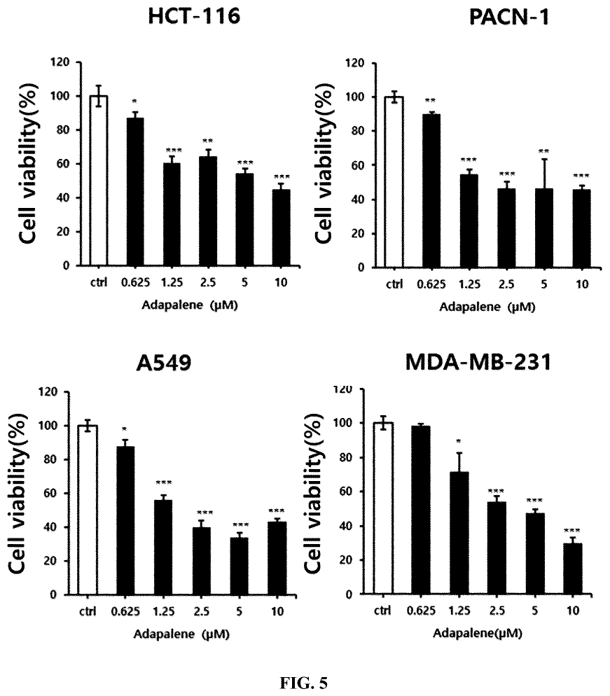

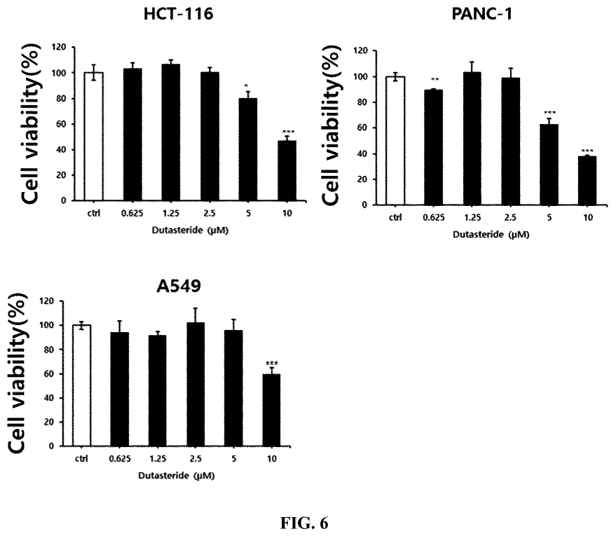

Ivacaftor showed a significant decrease in cell proliferation of pancreatic cancer cells (PANC-1), lung cancer cells (A549), and breast cancer cells (MDA-MB-231) (FIG. 4). Adapalene showed a significant decrease in cell proliferation of all of colorectal cancer cells (HCT-116), pancreatic cancer cells (PANC-1), lung cancer cells (A549), and breast cancer cells (MDA-MB-231) (FIG. 5). In addition, dutasteride showed a decrease in cell proliferation of colorectal cancer cells (HCT-116), pancreatic cancer cells (PANC-1), and lung cancer cells (A549) (FIG. 6).

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

The present invention aims at developing a new effective therapeutic agent for intractable cancer, which is difficult to radically treat previously and has a high recurrence rate. Currently, there are no effective targeted therapeutic agents or anticancer drugs for cancer, and research thereon is insufficient, and thus there is a need to develop new therapeutic agents. The present invention is for effective treatment of cancer using the expression level of a specific gene in a biological sample to be tested. According to the present invention, the PIP4K2C gene as a new target for cancer treatment has been discovered for the first time, and it has been found that when the expression of PIP4K2C is inhibited, breast cancer, lung cancer, pancreatic cancer, colon cancer, and rhabdomyosarcoma cell lines are efficiently killed, indicating that various PIP4K2C inhibitors may be very effectively used for the treatment of cancer patients as therapeutic compositions for radical treatment of cancer.

1-13. (canceled)

14. A method for preventing or treating cancer comprising a step of administering a PIP4K2C inhibitor as an active ingredient to a subject.

15. The method according to claim 14, wherein the cancer is selected from the group consisting of ovarian cancer, rhabdomyosarcoma, colorectal cancer, pancreatic cancer, gastric cancer, liver cancer, breast cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, hematological cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, cancer of ureter, renal cell carcinoma, renal pelvic carcinoma, central nervous system (CNS) tumor, primary CNS lymphoma, spinal cord tumor, brainstem glioma, and pituitary adenoma.

16. The method according to claim 14, wherein the PIP4K2C inhibitor comprises an antibody or antigen-binding fragment thereof that specifically binds to PIP4K2C, an aptamer that specifically binds to PIP4K2C, a nucleic acid molecule that inhibits expression of a nucleotide encoding PIP4K2C, or a small molecule compound that inhibits expression of PIP4K2C.

17. The method according to claim 16, wherein the PIP4K2C inhibitor further comprises at least one small molecule compound selected from the group consisting of dutasteride, aprepitant, ivacaftor, adapalene, and pharmaceutically acceptable salts thereof.

18. A method for screening a composition for preventing or treating cancer comprising steps of:
(a) bringing a test substance into contact with a biological sample containing cells expressing PIP4K2C; and
(b) measuring an activity or expression level of PIP4K2C or measuring a viability or proliferation rate of the cells expressing PIP4K2C,
wherein, when the activity or expression level of PIP4K2C decreases or when the viability or proliferation rate of the cells expressing PIP4K2C decreases, the test substance is determined to be a composition for preventing or treating cancer.

19. The method according to claim 18, wherein the biological sample is selected from the group consisting of whole blood, leukocytes, peripheral blood mononuclear cells, leukocyte buffy coat, plasma, serum, sputum, tears, mucus, nasal washes, nasal aspirate, breath, urine, semen, saliva, peritoneal washings, pelvic fluids, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, tissue, tissue-derived cells, cell extract, and cerebrospinal fluid.

20. A composition for diagnosing cancer comprising, as an active ingredient, an agent for measuring an expression level of PIP4K2C protein or a gene encoding the same.

21. The composition according to claim 20, wherein the agent for measuring the expression level of the PIP4K2C protein is an antibody or antigen-binding fragment thereof that specifically binds to the PIP4K2C protein, an aptamer that specifically binds to PIP4K2C, or a small molecule compound that inhibits expression of PIP4K2C.

22. The composition according to claim 20, wherein the agent for measuring the expression level of the gene encoding the PIP4K2C protein is a primer or probe that specifically binds to a nucleic acid molecule of the gene.
